(19) United States
(12) Patent Application Publication
(10) Pub. No.: US 2025/0195836 A1
(43) Pub. Date: Jun. 19, 2025

(54) REDUCED CONTACT-FRICTION TUBULAR MEMBER FOR INTRODUCING A MEDICAL DEVICE INTO A BODY

(71) Applicant: BOSTON SCIENTIFIC SCIMED, INC., MAPLE GROVE, MN (US)

(72) Inventors: John Mims, Austin, TX (US); Vladimir Mitelberg, Austin, TX (US); Stephen West, Austin, TX (US); Thomas Neudeck, Austin, TX (US)

(73) Assignee: BOSTON SCIENTIFIC SCIMED, INC., MAPLE GROVE, MN (US)

(21) Appl. No.: 18/980,355

(22) Filed: Dec. 13, 2024

Related U.S. Application Data
(60) Provisional application No. 63/610,758, filed on Dec. 15, 2023.

(51) Int. Cl. A61M 25/01

(52) U.S. Cl. CPC A61M 25/01

(57) ABSTRACT

At least a portion of the interior surface of a tubular member of an introducer for medical devices, such as an overtube, is provided with structural surface features to reduce contact friction between at least a portion of the interior surface of the tubular member and the exterior surface of a medical device longitudinally translated therethrough. The distal end of the introducer may be reinforced with a wire with flattened sides extending circumferentially around the lumen of the introducer so as not to impact the inner diameter of the lumen and thereby to allow simultaneous passage of one or more medical devices therethrough.

### CROSS-REFERENCE TO RELATED APPLICATIONS

This application claims the benefit of priority under 35 U.S.C. § 119 to U.S. Provisional Application No. 63/610,758, filed Dec. 15, 2023, the entire disclosure of which is hereby incorporated by reference herein for all purposes.

### FIELD

The present disclosure relates generally to the field of introducer devices, such as overtubes, for introducing a medical device into a patient's body, and related systems and methods. More particularly, the present disclosure relates to reduction of contact friction between an introducer device and a medical device advanced therethrough, such as to be advanced into a patient's body.

### BACKGROUND

Various medical devices, such as medical scopes (e.g., endoscopes) as well as associated mounted devices, may be introduced or inserted into a patient's body with the assistance of (e.g., through a lumen defined through) an introducer device, such as an overtube. In procedures in which a medical scope may be removed and re-introduced multiple times, the introducer device facilitates the passing of the medical device, including one or more devices mounted on the medical device, therethrough. However, repeated translation of a medical device through an introducer can create significant friction, and can increase device insertion and retraction forces. For instance, if a lubricant is used to facilitate passage of a medical device through an introducer, the lubricant may be wiped away by repeated relative movement between the medical device and the introducer. Excessive translational forces can result in inadvertent positional changes of the devices, which may require corrective action. Excessive insertion forces may present a safety concern, may compromise positional accuracy of a medical scope and/or associated devices, and/or could delay the procedure. This effect can be more pronounced if the introducer has a narrow distal end or “nose cone”. Nose cones, which may be made of a soft material, like silicone or low durometer polyurethane, may be used to facilitate intubation and/or to provide an effective seal with respect to a tubular medical device used for passage of materials (e.g., fluids such as liquids or gases) to minimize leakage (such as to maintain insufflation achieved with the use of the medical device). Often a water-based lubricant or saline might be added to the outside of the medical device to reduce frictional insertion and retraction forces. If the introducer nose-cone inner diameter is of an equal or lesser diameter than the outer diameter of the medical device outer (or the total outer diameter of a medical device with an outside mounted device like an overmounted system or a scope cap), the added lubricant can be wiped off from the outer surface of the inserted device and, especially upon retraction, may reduce or even eliminate the reduced-friction benefit to be imparted by the lubricant. In extreme cases, if the inserted device is significantly larger than the smallest inner diameter of a tapered nose-cone and causes the nose cone to stretch, the increase in surface contact area and stripping of fluids or lubricants from this interface can create a “suction cup effect” and increase friction to the point where the device can no longer be safely advanced or retracted relative to the introducer. Instead, the introducer and the inserted device would then need to be removed together from the patient to be safely separated, thereby defeating the purpose of using an introducer. Solutions to these and other challenges in the art would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system, configured for insertion within a patient's body, includes an introducer including a tubular elongate member having a wall defining a lumen extending through the introducer; and a medical device including an elongate member configured for insertion through the lumen of the introducer. In some aspects, one or more structural surface features are provided on at least a portion of an interior surface along the lumen of the introducer, the structural surface features being configured and/or positioned to reduce contact friction between the interior surface of the introducer and an exterior surface of the medical device extended therethrough.

In some aspects, the system further includes an auxiliary medical device mounted along the exterior surface of the medical device. In some aspects, the medical device includes a flexible elongate member, and the auxiliary medical device extends along an exterior surface of the flexible elongate member of the medical device.

In some aspects, the structural surface features are provided on at least an interior surface of a distal end of the tubular elongate member of the introducer.

In some aspects, the introducer includes a nose cone at a distal end of the tubular elongate member, the structural surface features being provided on an interior surface of the nose cone.

In some aspects, the nose cone is dimensioned to maintain a tight fit with respect to the exterior of the medical device. In some aspects, the system further includes an auxiliary medical device mounted along the exterior surface of the medical device; and the nose cone is dimensioned to maintain a tight fit with respect to the exterior of the medical device as well as with respect to an exterior of the auxiliary medical device.

In some aspects, at least one of the structural surface features comprise one or more shapes selected from the group consisting of: a rib, a groove, a bump, a dimple, or a saddle.

In some aspects, the structural surface features have uniform and/or varied heights, depths, and/or spacing therebetween.

In some aspects, at least a distal end of the introducer is reinforced with a flattened wire to resist kinking when being bent within a tortuous pathway and/or when being retroflexed to allow simultaneous passage of one or more medical devices through the lumen of the introducer without impacting the size of the inner diameter of the lumen of the introducer.

In accordance with various principles of the present disclosure, an introducer for a medical device includes a tubular elongate member defining a lumen therethrough; and one or more structural surface features on at least a portion of an interior surface of the lumen. In some aspects, the structural surface features are configured and positioned to reduce contact friction between the lumen and an exterior surface of one or more medical devices extended therethrough.

In some aspects, the structural surface features are provided on at least an interior surface of a distal end of the tubular elongate member of the introducer. In some aspects, the introducer includes a nose cone at a distal end of the tubular elongate member, the structural surface features being provided on an interior surface of the nose cone. In some aspects, the nose cone is formed separately from the tubular elongate member. In some aspects, at least one of the structural surface features comprise one or more shapes selected from the group consisting of: a rib, a groove, a bump, a dimple, or a saddle. In some aspects, the structural surface features have uniform and/or varied heights, depths, and/or spacing therebetween.

In accordance with various principles of the present disclosure, a method is disclosed for forming an introducer having a lumen sized, shaped, configured, and/or dimensioned for one or more medical device to be introduced therethrough and into a patient's body without interference by contact-friction with the interior surface of the lumen. In accordance with various principles of the present disclosure, the method includes providing one or more structural surface features along at least a portion of an interior surface of the lumen, the one or more structural surface features being configured and/or positioned to reduce contact friction between the interior surface of the introducer and an exterior surface of one or more medical devices extended through the introducer.

In some aspects, the method further includes forming at least one of the structural surface features as a shape selected from the group consisting of: a rib, a groove, a bump, a dimple, or a saddle; and forming the structural surface features with uniform and/or varied heights, depths, and/or spacing therebetween,

In some aspects, the method further includes reinforcing at least a distal end of the introducer with a flattened wire to resist kinking when being bent within a tortuous pathway and/or when being retroflexed without impacting the size of the inner diameter of the lumen of the introducer to allow simultaneous passage of one or more medical devices therethrough.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of a system with an introducer formed in accordance with various principles of the present disclosure.

FIG. 2A illustrates a perspective view of a portion of a distal end of an introducer formed in accordance with various principles of the present disclosure.

FIG. 2B illustrates a perspective view of a portion of a distal end of an introducer formed in accordance with various principles of the present disclosure, and with a distal end of an endoscope extending therethrough.

FIG. 2C illustrates a perspective view of a portion of a distal end of an introducer formed in accordance with various principles of the present disclosure, and with a distal end of an endoscope and an auxiliary device, extending along the exterior of the endoscope, extending therethrough.

FIG. 3 illustrates a distal perspective view of an example of an embodiment of a nose cone for an introducer formed in accordance with various principles of the present disclosure.

FIG. 4 illustrates a distal perspective view of an example of an embodiment of a nose cone for an introducer formed in accordance with various principles of the present disclosure.

FIG. 5 illustrates a distal perspective view of an example of an embodiment of a nose cone for an introducer formed in accordance with various principles of the present disclosure.

FIG. 6 illustrates a distal perspective view of an example of an embodiment of a nose cone for an introducer formed in accordance with various principles of the present disclosure.

FIG. 7 illustrates a distal perspective view of an example of an embodiment of a nose cone for an introducer formed in accordance with various principles of the present disclosure.

FIG. 8 illustrates an elevational view of an overtube formed in accordance with various principles of the present disclosure.

FIG. 8A illustrates a cross-sectional view along line VIIIA-VIIIA of FIG. 8.

FIG. 8B illustrates a detail view along detail area VIIIB in FIG. 8A.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure relates to improvements to generally tubular devices for introducing a medical device into the body of a patient, such as introducers such as overtubes. For instance, the introducer may be configured for insertion into a body passage, such as naturally-existing passages (e.g., the small intestine, the large intestine, the stomach, etc.) as well as medically-created passages (e.g., a passage created with the use of a medical instrument, and not existing without medical intervention) or otherwise. It will be appreciated that terms such introducer or overtube may be used interchangeably herein without intent to limit, reference being made herein to an overtube for the sake of convenience and without intent to limit.

An overtube formed in accordance with various principles of the present disclosure includes a tubular elongate member defining a lumen therethrough. In some aspects, the overtube is formed from any suitable biocompatible material having sufficient flexibility to traverse non-straight or tortuous anatomies, such as known to one of ordinary skill in the art. Such materials include, but are not limited to, rubber, silicon, synthetic plastic, stainless steel, metal-polymer composite; metal alloys of nickel, titanium, copper cobalt, vanadium, chromium, and iron; superelastic or shape memory material such as nitinol (nickel-titanium alloy); different layers of different materials and reinforcements. Such materials may be made of or coated with a polymeric or lubricious material to enable or facilitate passage of a delivery device therethrough. In some embodiments, the lumen therethrough may be made of or coated with a polymeric or lubricious material to facilitate passage of the introduced medical instrument(s) therethrough.

In accordance with various principles of the present disclosure, at least a portion of a lumen defined through the tubular elongate member of the overtube is modified to facilitate longitudinal translation of one or medical devices therethrough, such as repeated extension and retraction of the one or more medical devices through the lumen of the overtube. In some aspects, at least a portion of the interior surface of the wall of the tubular elongate member defining the lumen is modified. In some aspects, at least a distal end of the overtube is modified, such as the interior surface of a distal region of the tubular elongate member of the overtube. The one or more medical devices may include an elongate member configured for insertion through the lumen of the overtube, and one or more externally mounted devices (e.g., mounted longitudinally along the exterior of the flexible elongate member). In some aspects, the overtube and the one or more medical devices are sufficiently flexible to be navigated within tortuous body passages within a patient. It will be appreciated that the elongate member of the one or more medical devices translated within a lumen of the overtube may include be a flexible elongate member capable of being navigating through tortuous body passages. In some aspects, the flexible elongate member is a flexible elongate tubular member, including, without limitation, a medical scope (e.g., an endoscope). Other medical devices, such as auxiliary medical devices, may extend along the exterior of the elongate member of the inserted medical device for translation within the lumen of the overtube. The auxiliary medical device may include a flexible elongate member capable of being navigating through tortuous body passages. Examples of auxiliary medical devices include, without limitations, those described in U.S. Pat. No. 11,051,800 to Mitelberg et al., which issued on Jul. 6, 2021; and U.S. Provisional Patent Application ______ [Attorney Docket 2001.3236100], filed on May 4, 2023, which are hereby incorporated herein by reference in their entireties and for all purposes. The one or more medical devices may be inserted into the lumen of the overtube for longitudinal translation therein, and may alternatively be referenced as inserted devices for the sake of convenience and without intent to limit. It will further be appreciated that references may be made interchangeably herein to medical tools, instruments, or devices without intent to limit.

In accordance with various principles of the present disclosure, the interior surface of at least the distal end of an overtube is modified to adjust and/or to control the contact surface area between the interior of the overtube and the exterior of one or more medical devices longitudinally extended through the overtube (e.g., through the lumen defined through overtube). More particularly, at least the interior surface of the distal end of the overtube (e.g., the distal surface of the lumen of the overtube) is modified to minimize friction and/or to lower insertion and retraction forces between the overtube and one or more medical devices longitudinally translated therethrough. Alternatively or additionally, the at least a portion of the interior surface may be modified to minimize any negative effects the overtube might have on lubrication added between the overtube and the one or more medical devices longitudinally translating therethrough. Alternatively or additionally, the at least a portion of the interior surface of the overtube may be modified to prevent the creation of a uniform surface or edge that may wipe lubrication from the exterior surface of a medical device longitudinally translated through the lumen of the overtube. In some aspects, structural surface features are added or formed on the interior surface of at least the distal end of the overtube, such as an interior surface in direct contact with the introduced device. Such structural surface features are configured and/or positioned to reduce the contact surface area between the interior surface of the overtube (e.g., the surface of the lumen of the overtube) and the exterior surface of the inserted device.

Various embodiments of introducers formed in accordance with various principles of the present disclosure and associated devices and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 1000 are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments.

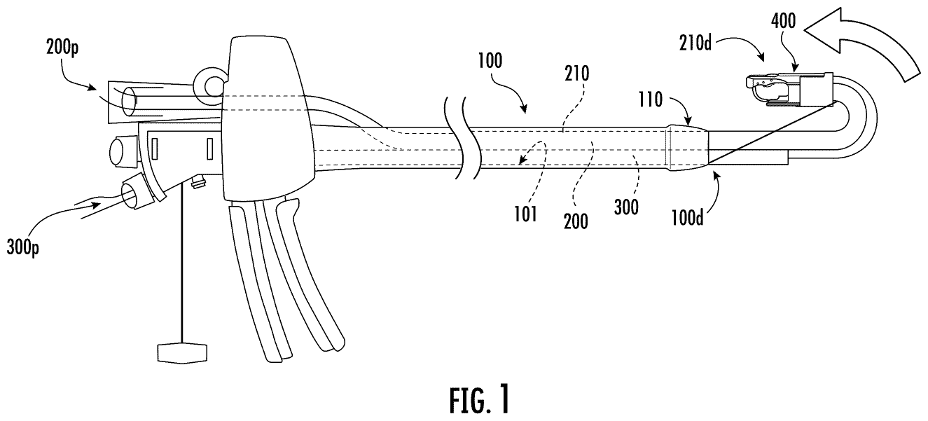

Turning now to the drawings, an example of an embodiment of an overtube 100 formed in accordance with various principles of the present disclosure is illustrated in FIG. 1 with a medical device 200 extended through the lumen 101 of the overtube 100. More particularly, at least a flexible elongate member 210 of the medical device 200 is extended through the lumen 101 of the overtube 100. In some aspects, the flexible elongate member 210 may be capable of being navigated, and, in some instances, the distal end 210d of the flexible elongate member 210 may be capable of retroflexion, such as illustrated in FIG. 1. In addition to the flexible elongate member 210 of the medical device 200 extending through the lumen 101 of the overtube 100, an auxiliary medical device 300 may be extended through the lumen 101 along the exterior of the medical device 200. In the example of an embodiment illustrated in FIG. 1, the auxiliary medical device 300 may include a control mechanism, such as a transmission element, extending to a device 400 positioned (e.g., mounted) with respect to the distal end 210d of the flexible elongate member 210. An auxiliary medical device 300 in the form of a control mechanism may allow for proximal control (e.g., by a medical professional and/or automated system) of the device 400 along a proximal end 300p of the auxiliary medical device 300, such as along a proximal end 210p of the medical device 200 outside the patient's body, such as to navigate and/or retroflex at least the distal end 210d of the flexible elongate member 210, such as schematically illustrated in FIG. 1 In some aspects, the auxiliary medical device 300 and the device 400 may be considered to be part of an end effector system, such as, without limitation, a tissue suturing system. Any of a variety of control handles may be provided along the proximal end 200p of the medical device 200 and/or along the proximal end 300p of the auxiliary medical device 300. As may be appreciated, the extension of an auxiliary medical device 300 along the exterior of the flexible elongate member 210 (in contrast with within a lumen defined through the flexible elongate member 210) may add to the overall cross-sectional size of the inserted devices, increasing some of the potential frictional issues described above. Additional medical devices may also be extended through the lumen 101 of the overtube 100, potentially creating further frictional issues. Various principles of the present disclosure address such potential frictional issues, such as described herein.

In accordance with various principles of the present disclosure, the interior surface of at least the distal end 100d of the overtube 100 is modified from the typically smooth interior of currently available overtubes. Modifications to at least a portion of the interior surface of the overtube 100 are positioned and configured in accordance with various principles of the present disclosure to reduce the contact friction between at least a portion of the interior of the overtube 100 and the exterior of the one or more medical devices 200, 300 longitudinally translating within the lumen 101 of the overtube 100. For the sake of convenience, and without intent to limit, reference is made simply to the interior of the overtube 100 as encompassing at least the interior of the distal end 100d thereof and/or interior surfaces proximal thereto, whether or not continuous/coextensive with the interior of the distal end 100d of the overtube 100. The modified interior surface encompasses roughening or coating of the surface as well as more pronounced structural modifications. More pronounced structural modifications may include formation of structural surface features which are more readily distinguishable from surface roughening or coatings (e.g., distinguishable visibly by an unaided human eye, or by manual tactile inspection, such as by a distinct change in shape or structure relative to surrounding surfaces). For instance, in some aspects, a structural surface feature imparts a macroscopic affect which is greater than a microscopic affect imparted by surface roughening and/or coatings. Alternatively or additionally, a tactile structural surface feature in accordance with various principles of the present disclosure may have differentiable surface features which may be felt by a user's hands or fingers as distinct elements (e.g., spaced apart raised or indented elements, in contrast with a smaller scale surface roughening). As may be appreciated by those of ordinary skill in the art, the dimensions are selected to impact the surface areas of the overtube 100 and medical devices 200, 300, etc., in contact with each other to reduce the resultant contact friction therebetween, as may be determined by one of ordinary skill in the art.

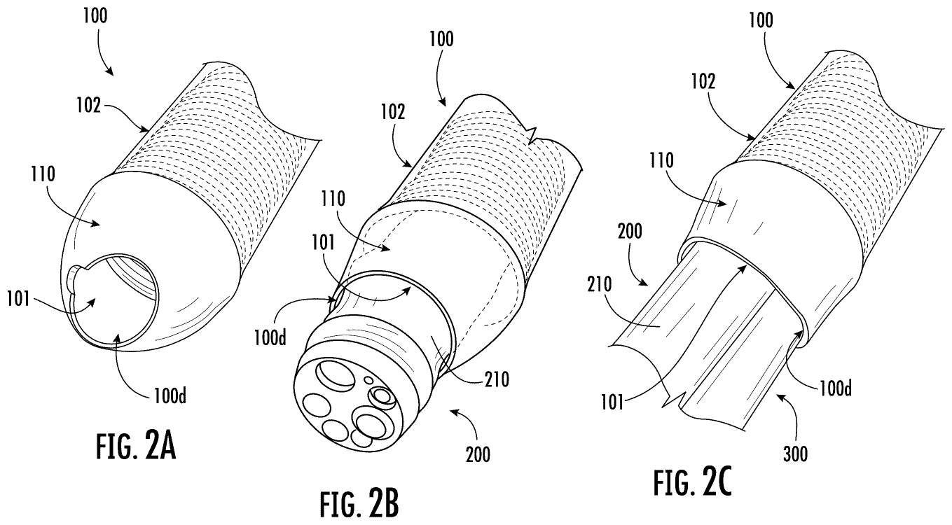

It will be appreciated that although the overtube 100 may have a generally constant cross-sectional dimension (e.g., generally constant outer diameter and/or inner diameter) along a tubular elongate member 102 thereof, in some embodiments it may be desirable to taper the distal end 100d of the overtube 100, such as illustrated in FIG. 2A, FIG. 2B, and FIG. 2C. A tapered distal end 100d may facilitate insertion of the overtube 100 into a patient and/or may provide a secure/tight fit with respect to the exterior surface of the medical devices 200, 300, etc., therein. The tapered distal end 100d may alternately be referenced as a nose cone 110 for the sake of convenience and without intent to limit. It will be appreciated that references herein to a tapered distal end of an overtube are intended to encompass a nose cone, if provided, whether or not the nose cone is formed with or separately from the remaining portions of the overtube.

It will be appreciated that the tapered distal end 100d of the overtube 100 may be generally monolithic with the proximal portions of the overtube 100, such as the tubular elongate member 102. However, in some embodiments, the tapered distal end 100d of the overtube 100 may be a separately formed element from the tubular elongate member 102, which may have a more uniform cross-sectional dimension along its length (a generally uniform inner diameter and/or outer diameter), such as to facilitate manufacture thereof. The separately formed tapered distal end 100d may be coupled with the distal end of the tubular elongate member 102 of an overtube 100 in any of a variety of manners. Preferably, such coupling is secure and/or airtight. Manners of coupling include, without limitation, adhering (e.g., with glue), fusing, welding (e.g., ultrasonic, or otherwise), crimping, etc., to the elongate member 102 of the overtube 100. In some aspects, the tapered distal end 100d and the more uniform portion of the overtube 100 may be formed at the same time, such as by dipping, coating, spraying, etc., the material from which the overtube 100 is formed over an appropriately shaped mandrel or the like. It will be appreciated that the present disclosure need not be limited in this regard.

In embodiments in which the distal end 100d of the overtube 100 is tapered, structural surface features such as described above may be limited to the tapered portion, or may extend proximally, to portions of the overtube 100 proximal to the tapered distal end 100d, as well. For the sake of convenience, reference is made herein to provision of structural surface features to the interior surface of the nose cone 110 of an overtube 100. However, such references are intended to include application to the interior surface of additional portions of the overtube 100 and/or to interior surfaces of overtubes 100 without a tapered distal end 100d, unless otherwise explicitly stated.

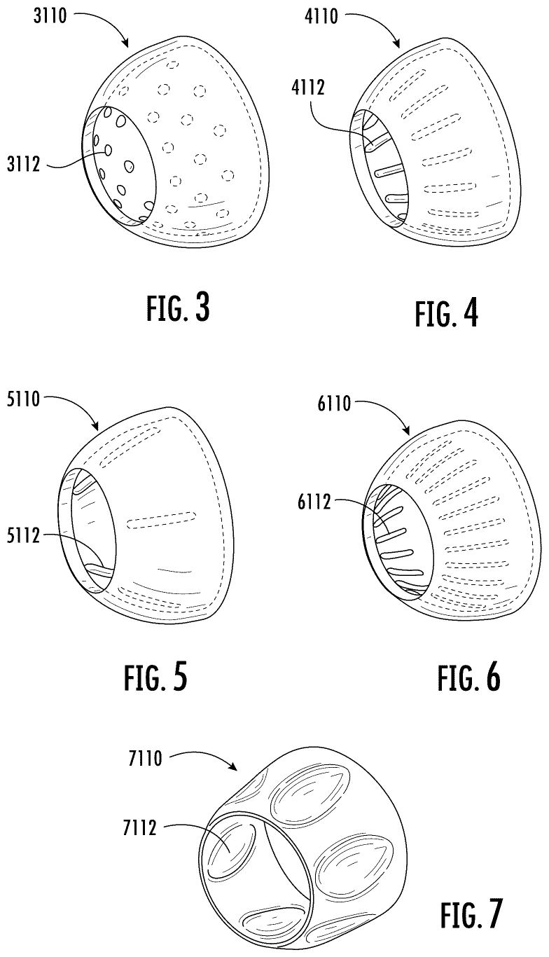

In accordance with various principles of the present disclosure, structural surface features provided on the interior of the nose cone 110 may be in the form of protruding shapes (e.g., raised above the surrounding surface) such as longitudinal ribs and/or bumps, and/or recessed shapes (e.g., indented or depressed into the surface) such as grooves and/or dimples. The ribs/grooves need not be straight, and, instead, may be at least somewhat curved (e.g., helical) if desired. The bumps/dimples may be circular or any other acceptable shape, as may readily be determined by one of ordinary skill in the art. Such structural surface features could be of various heights, depths, and/or frequencies (e.g., spacing, such as at regular or irregular intervals) to optimize performance based on the contacting surfaces, material, and/or clinical application. Non-limiting examples of structural surface features are illustrated in FIGS. 3-7, as will now be described. It will be appreciated that the various embodiments of overtube sections/nose cones illustrated in FIGS. 3-7 have many features in common with one another, as well as with the overtube/nose cone 110 illustrated in FIG. 1. Accordingly, common features are identified by common reference elements differing by multiples of 1000, and, for the sake of brevity, the descriptions of the common features will not necessarily be repeated. For purposes of clarity, not all components having the same reference number are numbered.

In the example of an embodiment of a nose cone 3110 illustrated in FIG. 3, structural surface features 3112 in the form of protruding bumps and/or recessed dimples are provided along the interior surface. Although the structural surface features 3112 are illustrated as generally uniformly spaced apart, the present disclosure need not be limited in this regard.

In the example of an embodiment of a nose cone 4110 illustrated in FIG. 4, structural surface features 4112 in the form of ribs and/or grooves are provided along the interior surface. Although the structural surface features 4112 are illustrated as generally uniformly spaced apart, the present disclosure need not be limited in this regard. Moreover, the spacing between the ribs and/or grooves and/or the length (e.g., relative to the length of the nose cone) may be varied from that illustrated in FIG. 4. For example, in the example of an embodiment of a nose cone 5110 illustrated in FIG. 5, the rib/groove structural surface features 5112 are longer and more widely spaced apart than the rib/groove structural surface features 4112 of the nose cone 4110 illustrated in FIG. 4. And, in the example of an embodiment of a nose cone 6110 illustrated in FIG. 6, the rib/groove structural surface features 6112 are shorter and more closely spaced apart than the rib/groove structural surface features 4112 of the nose cone 4110 illustrated in FIG. 4. It will be appreciated that other variations of spacing and/or length are within the scope of the present disclosure, the present disclosure not being limited by the illustrated examples of embodiments. Moreover, the spacing and/or length need not be uniform among structural surface features of a nose cone.

In the example of an embodiment of a nose cone 7110 illustrated in FIG. 7, structural surface features 7112 in the form of shapes extending generally longitudinally along the nose cone 7110 and generally wider than the ribs/grooves of FIGS. 4-6 are provided along the interior surface of the nose cone 7110. Such structural surface features 7112 may be referenced herein as “saddles”. The configuration of the saddles 7112 is selected to reduce the contact of the interior of the nose cone 7110 with the exterior of devices 200, 300, etc., inserted through the nose cone 7110, thereby avoiding such adverse effects of prior overtubes described above. For instance, the saddles 7112 may protrude from the interior of the nose cone 7110 to reduce the contact surface area between the nose cone 7110 and devices 200, 300, etc., extended through the nose cone 7110. Conversely, recessed saddles 7112 depressed into the interior surface of the nose cone 7110 would also reduce the overall contact surface area between the nose cone 7110 and devices 200, 300, etc., extended through the nose cone 7110. In some aspects, the distance between the surface of the saddles 7112 and the surrounding surface of the nose cone 7110 is more gradual axially and/or radially than at least some of the other structural surface features described herein. This may help to reduce the likelihood of lubricant transfer at the saddle when the scope is moved axially and torqued. A saddle feature such as saddles 7112 can also be utilized to adjust/increase the stiffness of a nose cone 7110 (or the like) beyond what can be achieved with a compliant material with straight/constant thickness wall to reduce the risk of the nose cone prolapsing/inverting when retracting a large device. Although the saddles 7112 are wider/larger than the above-described bumps, dimples, ribs, grooves, the dimensions are selected so that the contact surface area between the nose cone 7110 and devices 200, 300, etc., extended through the nose cone 7110 does not cause a deleterious amount of contact friction.

It will be appreciated that dimensions (radial and/or circumferential), spacing, etc., of surface features on the interior surface of a nose cone such as described herein may be determined by those of ordinary skill in the art with considerations such as the material and/or material properties and/or dimensions of the nose cone and/or the inserted devices. For instance, various factors, such as the durometer (e.g., softness) of the material from which the overtube and/or nose cone are formed, the manufacturing technique used to form the overtube, the surface properties (e.g., lubricity) of the inserted devices, etc., may affect the selection of the positions, sizes, shapes, configurations, and/or dimensions of the structural surface features.

Structural surface features such as described herein may be provided on an interior surface of at least a portion of an overtube (e.g., along a nose cone thereof or coupled thereto and generally considered a portion thereof) in any of a variety of manners, such as, without limitation, those known to those of ordinary skill in the art. For instance, structural surface features may be overmolded, heat formed, fused, or otherwise added to a portion of an interior surface of an already-formed overtube. Alternatively or additionally, structural surface features may be formed on an interior surface of at least a portion of an overtube during the manufacture of the overtube. For instance, an overtube (and/or a separately formed nose cone) may be formed by a molding process with the structural surface features formed during formation of the overtube/nose cone. Alternatively or additionally, a liquified form of the material from which the overtube (and/or separately formed nose cone) is to be formed may be a dipped or sprayed over a mandrel configured to form the structural surface features along an interior surface thereof.

The material from which the overtube (and/or separately formed nose cone) is made is typically resilient and/or elastic (e.g., stretchable), such as to allow passage of an inserted device out the open distal end thereof while still remaining in physical direct contact with the exterior surface of the inserted device. For instance, the overtube (and/or separately formed nose cone) may be formed of silicone, low durometer polyurethane, and/or other materials (preferably biocompatible materials) commonly used for medical overtubes or introducers. As such, if formed over a mandrel, the overtube may be expanded to be removed from the mandrel and any protrusions on the mandrel forming recessed structural surface features.

Returning again to FIG. 2B and FIG. 2C the illustrated example of an embodiment overtube 100, or at least a distal end 100d thereof, is formed of a flexible stretchable material to allow expansion of the open distal end 100d thereof to allow distal extension of medical device 200, 300, etc., therethrough. And, the material of the overtube 100 may have sufficient resiliency to seal around the exterior of the medical devices 200, 300, etc. For instance, in the example of an embodiment illustrated in FIG. 2B, a flexible elongate member 210 of a medical device 200 in the form of an endoscope is inserted through the lumen 101 of the overtube 100 and distally through the distal end 100d of the overtube 100 in the form of a nose cone 110, expanding the open distal end 100d of the overtube 100. The nose cone 110 contracts against the exterior of the flexible elongate member 210 (e.g., an insertion tube) of the medical device 200. In the example of an embodiment illustrated in FIG. 2C, a medical device 200 along with an auxiliary medical device 300 are extended through the lumen 101 of the overtube 100 and distally through the open distal end 100d of the overtube 100. The nose cone 110 contracts against the exterior of the flexible elongate member 210 (e.g., an insertion tube) of the medical device 200 as well as against the exterior of the auxiliary medical device 300.

In some aspects, sealing of the overtube 100 with respect to the medical device 200 may be helpful to maintain a desired environment, such as created by the medical device 200. For instance, in the case of an endoscope used to insufflate a target area within the patient's body, it would be desirable to maintain a given insufflation pressure within the patient's body. Accordingly, a tight seal between the interior of the distal end 100d of the overtube 100 and the exterior of medical devices 200, 300, etc., inserted therethrough may be desirable. As may be appreciated with reference to FIG. 2C, an irregular exterior cross-dimensional shape of a medical device 200 with an auxiliary medical device 300 extending therealong may interfere with sealing between the devices 200, 300 and the interior of the distal end 100d of the overtube 100. In some embodiments, a sealing device, such as an inflatable bladder, may be provided around the inserted devices 200, 300 and/or on or within the overtube 100 to maintain a seal therebetween. The sealing device is configured and adapted to form a seal around the inserted devices 200, 300, such as to seal gaps which may be formed by an auxiliary medical device 300 extending along the exterior of the medical device 200, better than seals provided by a standard overtube or nose cone. For instance, a sealing device such as in the form of an inflatable bladder can conform to the shape of devices inserted therethrough to seal the interior of the overtube distal to the sealing device. With such seal, a medical professional need not insufflate the target area in the patient as often, thereby enhancing visualization during a procedure. In some embodiments, the sealing device is provided along the distal end 100d of the overtube 100, such as along the nose cone 110. In other embodiments, the sealing device is provided along the proximal end 100p of the overtube 100.

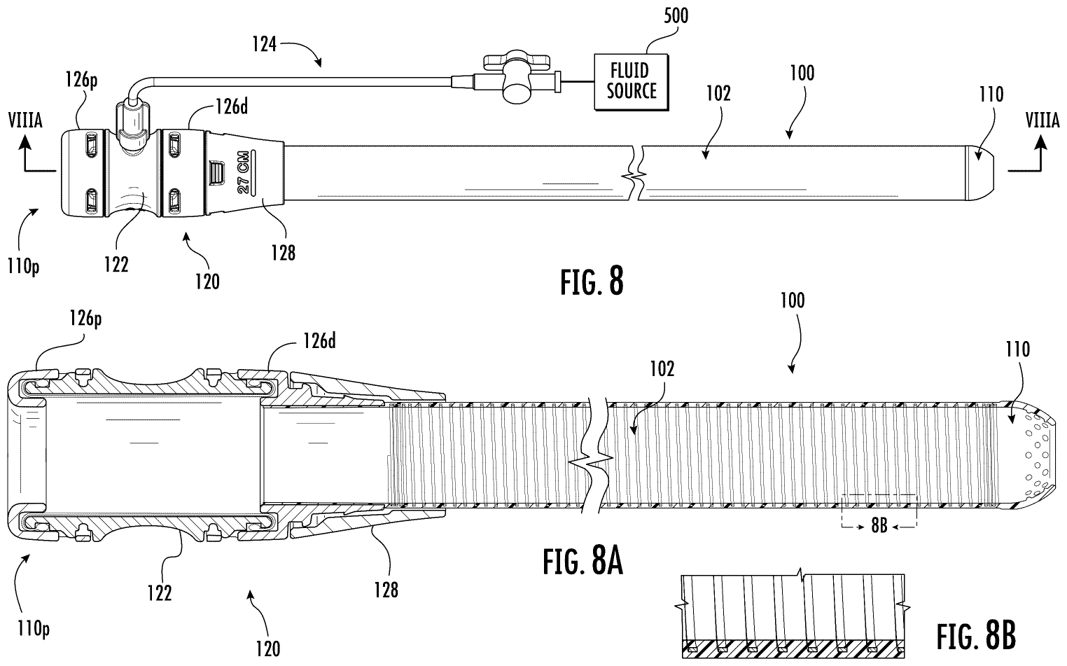

In the example of an embodiment of an overtube 100 illustrated in FIG. 8, a control handle 120 is provided along the proximal end 100p of the overtube 100, with a sealing device therein. More particularly, as may be appreciated with reference to FIG. 8 along with FIG. 8A, illustrating a cross-sectional view along line VIIIA-VIIIA in FIG. 8, the control handle 120 includes an inflatable cuff 122 fluidly coupled with a fluid source 500, such as via a stopcock subassembly 124. In some aspects, the fluid is air, although other fluids, such as water or saline, may be used. The inflatable cuff 122 may be formed of a resilient material capable of exerting pressure and sealing with respect to a device inserted through the inflatable cuff 122 upon inflation of the inflatable cuff 122. Suitable materials include, without limitation, polypropylene, silicone, rubber (e.g., Santoprene® vulcanized rubber), etc. The stopcock subassembly 124 and fluid source 500 may be any suitable stopcock assembly and fluid source 500 such as known to those of ordinary skill in the art, the present disclosure not being limited in this regard. The inflatable cuff 122 may be held in place with respect to the overtube 100 and medical devices 200, 300, etc., extended through the overtube 100 with a proximal collar 126p and a distal collar 126d. The inflatable cuff 122 and/or the distal collar 126d may be held with respect to the elongate member 112 of the overtube 100 with a locking collar 128. The collars 126p, 126d, and 128 may be any suitable collars known to those of ordinary skill in the art, the present disclosure not being limited in this regard.

In some aspects, an overtube formed in accordance with various principles of the present disclosure may have a reinforced region along a longitudinal and/or circumferential extent of at least a portion of the overtube. For instance, if an inserted device is flexible to be navigated within a patient's body and/or capable of retroflexion such as in the example of an embodiment illustrated in FIG. 1, it may be desirable to reinforce at least a portion the overtube to make it more kink resistant (e.g., so the lumen therein does not collapse upon bending, such as greater than 90° from a straight configuration during retroflexion, intubation, and/or passing through a tortuous anatomy). A kink-resistant region may be formed by providing a reinforcement element, such as a reinforcement wire, in or along the wall of the reinforced portion of the overtube. For instance, a wire coil may be provided with respect to (e.g., within the wall of) an overtube to reinforce a portion of a wall thereof, such as illustrated in FIG. 8A.

In accordance with various principles of the present disclosure, reinforcement of at least a portion of a wall of an overtube is modified to accommodate insertion of more than one medical device through the lumen of the overtube. More particularly, reinforcement of at least a portion of a wall of an overtube is modified in accordance with various principles of the present disclosure to allow for an increase in the size of the inner diameter of the lumen of the overtube without affecting the size of the outer diameter of the overtube. It will be appreciated that it may be desirable to maintain an outer diameter at a selected size determined by the body passage through which the overtube is anticipated to be inserted. As such, increases in the outer diameter of an overtube are generally undesirable. In accordance with various principles of the present disclosure, instead of using a reinforcement wire with a circular cross-section, as in prior art reinforced sections of tubular elements, a reinforcement wire with a generally flattened cross-sectional configuration (e.g., shortened in a radial direction, and optionally more elongated in a circumferential direction) is used to reinforce at least a portion of an overtube (e.g., a wall of an overtube), as illustrated in FIG. 8B, illustrating detail area VIIIB of FIG. 8A. In some embodiments, the pitch of the reinforcement wire may be increased or decreased to modify the kink resistance imparted by the reinforcement wire. In some aspects, the reinforcement wire may be formed of stainless steel, nitinol, polyether ether ketone (PEEK), etc.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. It should further be appreciated that the various separate features of the present subject matter need not all be present in order to achieve at least some of the desired characteristics and/or benefits of the present subject matter or such individual features. Therefore, the present invention is not limited to only the embodiments specifically described herein, and all substitutes and modifications apparent to those skilled in the art are deemed to be within the spirit, scope, and concept of the disclosure as defined by the appended claims. Moreover, various further benefits of the various aspects, features, components, and structures of overtubes such as described above, in addition to those discussed above, may be appreciated by those of ordinary skill in the art. Individual features of any embodiment may be used and can be claimed separately or in combination with features of that embodiment or any other embodiment, the scope of the subject matter being indicated by the appended claims, and not limited to the foregoing description.

In view of the above, it should further be understood that the various embodiments illustrated in the figures have several separate and independent features, which each, at least alone, has unique benefits which are desirable for, yet not critical to, the presently disclosed overtubes. It is to be understood by one of ordinary skill in the art that the present discussion is a description of illustrative examples of embodiments only, and is not intended as limiting the broader aspects of the present disclosure. Therefore, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system configured for insertion within a patient's body, said system comprising:
an introducer including a tubular elongate member having a wall defining a lumen extending through said introducer; and
a medical device including an elongate member configured for insertion through the lumen of said introducer;
wherein one or more structural surface features are provided on at least a portion of an interior surface along the lumen of said introducer, the structural surface features being configured and/or positioned to reduce contact friction between the interior surface of said introducer and an exterior surface of said medical device extended therethrough.

2. The system of claim 1, further comprising an auxiliary medical device mounted along the exterior surface of said medical device.

3. The system of claim 2, wherein said medical device includes a flexible elongate member, and said auxiliary medical device extends along an exterior surface of said flexible elongate member of said medical device.

4. The system of claim 1, wherein said structural surface features are provided on at least an interior surface of a distal end of said tubular elongate member of said introducer.

5. The system of claim 1, wherein said introducer includes a nose cone at a distal end of said tubular elongate member, said structural surface features being provided on an interior surface of said nose cone.

6. The system of claim 1, wherein said nose cone is dimensioned to maintain a tight fit with respect to the exterior of said medical device.

7. The system of claim 6, wherein:
said system further comprises an auxiliary medical device mounted along the exterior surface of said medical device; and
said nose cone is dimensioned to maintain a tight fit with respect to the exterior of said medical device as well as with respect to an exterior of said auxiliary medical device.

8. The system of claim 1, wherein at least one of said structural surface features comprise one or more shapes selected from the group consisting of: a rib, a groove, a bump, a dimple, or a saddle.

9. The system of claim 1, wherein said structural surface features have uniform and/or varied heights, depths, and/or spacing therebetween.

10. The system of claim 1, wherein at least a distal end of said introducer is reinforced with a flattened wire to resist kinking when being bent within a tortuous pathway and/or when being retroflexed to allow simultaneous passage of one or more medical devices through the lumen of said introducer without impacting the size of the inner diameter of the lumen of said introducer.

11. An introducer for a medical device, said introducer comprising:
a tubular elongate member defining a lumen therethrough; and
one or more structural surface features on at least a portion of an interior surface of the lumen;
wherein said structural surface features are configured and positioned to reduce contact friction between the lumen and an exterior surface of one or more medical devices extended therethrough.

12. The introducer of claim 11, wherein said structural surface features are provided on at least an interior surface of a distal end of said tubular elongate member of said introducer.

13. The introducer of claim 11, wherein said introducer includes a nose cone at a distal end of said tubular elongate member, said structural surface features being provided on an interior surface of said nose cone.

14. The introducer of claim 13, wherein said nose cone is formed separately from said tubular elongate member.

15. The introducer of claim 12, wherein at least one of said structural surface features comprise one or more shapes selected from the group consisting of: a rib, a groove, a bump, a dimple, or a saddle.

16. The introducer of claim 12, wherein said structural surface features have uniform and/or varied heights, depths, and/or spacing therebetween.

17. The introducer of claim 12, wherein at least a distal end of said tubular elongate member is reinforced with a flattened wire to resist kinking when being bent within a tortuous pathway and/or when being retroflexed to allow simultaneous passage of one or more medical devices through the lumen of said introducer without impacting the size of the inner diameter of the lumen of said introducer.

18. A method of forming an introducer having a lumen sized, shaped, configured, and/or dimensioned for one or more medical device to be introduced therethrough and into a patient's body without interference by contact-friction with the interior surface of the lumen, said method comprising providing one or more structural surface features along at least a portion of an interior surface of the lumen, the one or more structural surface features being configured and/or positioned to reduce contact friction between the interior surface of the introducer and an exterior surface of one or more medical devices extended through the introducer.

19. The method of claim 18, further comprising:
forming at least one of the structural surface features as a shape selected from the group consisting of: a rib, a groove, a bump, a dimple, or a saddle; and
forming the structural surface features with uniform and/or varied heights, depths, and/or spacing therebetween.

20. The method of claim 18, further comprising reinforcing at least a distal end of the introducer with a flattened wire to resist kinking when being bent within a tortuous pathway and/or when being retroflexed without impacting the size of the inner diameter of the lumen of the introducer to allow simultaneous passage of one or more medical devices therethrough.
